SEQUENCE LISTING (7 sequences)

SEQ ID NO: 1 - protein, 1440 aa, Homo sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHQAYRYRRGEITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 2 - protein, 2813 aa, Homo sapiens
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDVQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLCDLAPEAPPPTLPPDMAQVTVGPGLLGVSTLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVLQRCCSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICMDEDGNEKRPGDVWTLPDQCHTVTCQPDGQTLLKSHRVNCDRGLRPSCPNSQSPVKVEETCGCRWTCPCVCTGSSTRHIVTFDGQNFKLTGSCSYVLFQNKEQDLEVILHNGACSPGARQGCMKSIEVKHSALSVELHSDMEVTVNGRLVSVPYVGGNMEVNVYGAIMHEVRFNHLGHIFTFTPQNNEFQLQLSPKTFASKTYGLCGICDENGANDFMLRDGTVTTDWKTLVQEWTVQRPGQTCQPILEEQCLVPDSSHCQVLLLPLFAECHKVLAPATFYAICQQDSCHQEQVCEVIASYAHLCRTNGVCVDWRTPDFCAMSCPPSLVYNHCEHGCPRHCDGNVSSCGDHPSEGCFCPPDKVMLEGSCVPEEACTQCIGEDGVQHQFLEAWVPDHQPCQICTCLSGRKVNCTTQPCPTAKAPTCGLCEVARLRQNADQCCPEYECVCDPVSCDLPPVPHCERGLQPTLTNPGECRPNFTCACRKEECKRVSPPSCPPHRLPTLRKTQCCDEYECACNCVNSTVSCPLGYLASTATNDCGCTTTTCLPDKVCVHRSTIYPVGQFWEEGCDVCTCTDMEDAVMGLRVAQCSQKPCEDSCRSGFTYVLHEGECCGRCLPSACEVVTGSPRGDSQSSWKSVGSQWASPENPCLINECVRVKEEVFIQQRNVSCPQLEVPVCPSGFQLSCKTSACCPSCRCERMEACMLNGTVIGPGKTVMIDVCTTCRCMVQVGVISGFKLECRKTTCNPCPLGYKEENNTGECCGRCLPTACTIQLRGGQIMTLKRDETLQDGCDTHFCKVNERGEYFWEKRVTGCPPFDEHKCLAEGGKIMKIPGTCCDTCEEPECNDITARLQYVKVGSCKSEVEVDIHYCQGKCASKAMYSIDINDVQDQCSCCSPTRTEPMQVALHCTNGSVVYHEVLNAMECKCSPRKCSK

SEQ ID NO: 3 - protein, 567 aa, Homo sapiens
SLSCRPPMVKLVCPADNLRAEGLECTKTCQNYDLECMSMGCVSGCLCPPGMVRHENRCVALERCPCFHQGKEYAPGETVKIGCNTCVCQDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHQEPGGLVVPPTDAPVSPTTLYVEDISEPPLHQEPGGLVVPPTDAPVSPTTLYVEDISEPPLH

SEQ ID NO: 4 - protein, 1110 aa, Homo sapiens
SLSCRPPMVKLVCPADNLRAEGLECTKTCQNYDLECMSMGCVSGCLCPPGMVRHENRCVALERCPCFHQGKEYAPGETVKIGCNTCVCQDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLCDLAPEAPPPTLPPDMAQVTVGPGLLGVSTLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVLQRCCSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCS

SEQ ID NO: 5 - protein, 1365 aa, Homo sapiens
SLSCRPPMVKLVCPADNLRAEGLECTKTCQNYDLECMSMGCVSGCLCPPGMVRHENRCVALERCPCFHQGKEYAPGETVKIGCNTCVCQDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLCDLAPEAPPPTLPPDMAQVTVGPGLLGVSTLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVLQRCCSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICMDEDGNEKRPGDVWTLPDQCHTVTCQPDGQTLLKSHRVNCDRGLRPSCPNSQSPVKVEETCGCRWTCPCVCTGSSTRHIVTFDGQNFKLTGSCSYVLFQNKEQDLEVILHNGACSPGARQGCMKSIEVKHSALSVELHSDMEVTVNGRLVSVPYVGGNMEVNVYGAIMHEVRFNHLGHIFTFTPQNNEFQLQLSPKTFASKTYGLCGICDENGANDFMLRDGTVTTDWKTLVQEWTVQRPGQTCQPILE

SEQ ID NO: 6 - protein, 1142 aa, Homo sapiens
SLSCRPPMVKLVCPADNLRAEGLECTKTCQNYDLECMSMGCVSGCLCPPGMVRHENRCVALERCPCFHQGKEYAPGETVKIGCNTCVCQDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLCDLAPEAPPPTLPPDMAQVTVGPGLLGVSTLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVLQRCCSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICMDEDGNEKRPGDVWTLPDQCHTVTCQ

SEQ ID NO: 7 - protein, 16 aa, Homo sapiens
SFSQNSRHQAYRYRRG

(19) United States
(12) Patent Application Publication
(10) Pub. No.: US 2022/0305089 A1
(43) Pub. Date: Sep. 29, 2022

(54) STABILIZING BUFFER FOR FACTOR VIII AND VWF

(71) Applicant: OCTAPHARMA AG, Lachen (CH)

(72) Inventors: Ronny SCHMIDT, Schriesheim (DE); Tilo SCHWIENTEK, Neuss (DE)

(73) Assignee: OCTAPHARMA AG, Lachen (CH)

(21) Appl. No.: 17/633,836

(22) PCT Filed: Aug. 14, 2020
(86) PCT No.: PCT/EP2020/072919

§ 371 Date: Feb. 8, 2022

(30) Foreign Application Priority Data
Aug. 16, 2019 (EP) 19192137.8

(51) Int. Cl. A61K 38/37; A61K 47/02; A61K 47/18; A61K 47/26; A61K 47/42; A61K 9/19; A61K 9/08

(52) U.S. Cl. CPC A61K 38/37; A61K 47/02; A61K 47/183; A61K 47/26; A61K 47/42; A61K 9/19; A61K 9/08

(57) ABSTRACT

The invention relates to a pharmaceutical composition comprising an isolated Factor VIII protein and/or an isolated VWF protein in a stabilizing buffer composition, wherein said pharmaceutical composition is free of albumin and said stabilizing buffer 5 composition comprises cryoprotectants and bulking agents in a weight ratio of more than 0.65:1. The invention relates further relates to the use of a stabilizing buffer formulating an FVIII protein and/or a VWF protein to the use of a VWF protein for stabilizing a FVIII protein in vitro.

### FIELD OF THE INVENTION

The invention relates to a stabilizing buffer composition for stabilizing Factor VIII (FVIII) and/or von Willebrand factor (VWF) with a defined ratio of cryoprotectants to bulking agents and to the in vitro stabilization of FVIII by VWF.

### BACKGROUND OF THE INVENTION

Hemophilia is a group of hereditary genetic disorders that impair the body's ability to control blood clotting or coagulation. In its most common form, Hemophilia A, the function of clotting factor VIII (FVIII) is impaired. Human Hemophilia A occurs in about 1 in 5,000-10,000 male births. The FVIII protein is an essential cofactor in blood coagulation with multifunctional properties. Substitution therapy of Hemophilia A can be performed with plasma-derived concentrates of FVIII or with recombinantly produced FVIII. The treatment with state-of-the-art FVIII concentrates facilitates a normalized life of Hemophilia A patients.

Hemophilia A patients are treated with FVIII on demand or as a prophylactic therapy administered several times a week. For prophylactic treatment, 15-25 IU/kg bodyweight of FVIII is administered three times a week, which is necessary due to the constant need of FVIII and its short half-life in the blood system, which in humans is only about 11 hours (Ewenstein et al., 2004).

In the blood, under normal conditions, the FVIII molecule is associated with von Willebrand factor (VWF), which stabilizes the FVIII molecule from different forms of degeneration. The non-covalent complex of FVIII and VWF has a high binding affinity of 0.2-0.3 nM (Vlot et al., 1996).

In a pharmaceutical formulation of FVIII, all components need to be carefully selected. The excipients provide a protective function to keep a high yield of FVIII throughout the pharmaceutical production process, through long-term storage, and finally for reconstitution and administration to the patient. In addition, the clinical safety of all excipients is considered. FVIII is a relatively big and complex molecule, which contains both ionic and hydrophobic parts. Accordingly, the tertiary and quaternary structure of FVIII is affected by the salt concentration/osmolality of the stabilizing buffer composition. Unsuitable salt concentrations/osmolality negatively influence the structure of the

FVIII molecule leading to a decrease in stability.

FVIII is usually administered in form of a liquid pharmaceutical composition, e.g. by intravenous administration. For storage purposes, the liquid pharmaceutical compositions are generally lyophilized. The main purpose of the lyophilization procedure is to remove water from the formulation, since adverse physical and chemical reactions often take place in the aqueous phase (Manning, M. C. et al 1989, Tang, X. et al, 2004, Schwegman, J. J. et al, 2005).

Cryoprotectants may be added to protect the protein during the freeze-drying process and during storage, primarily by forming an amorphous matrix surrounding the protein. Bulking agents may be included to function as a “cake former” to give mechanical support during freeze-drying and to increase the dry weight of the drug product. The bulking agent thereby contributes to providing a uniform quality and appearance of the lyophilized product. Buffering agents may be added to maintain the pH to a value suitable for the protein and for therapeutic use of the reconstituted product. Wang W. et al (2000) provides a comprehensive overview of the lyophilization process and specifically the function of various excipients.

Because of the high potency of the FVIII protein, the concentration of FVIII in therapeutic solutions is low. In addition, FVIII easily adsorbs to surfaces, making surface adsorption a major source of protein and activity loss during manufacturing and after reconstitution of the product. For presently marketed FVIII products, it is usually stated that a surface-active agent is used below its critical micelle concentration (cmc), which is the solution concentration at which the surface-active agent starts to form micellar aggregates. The cmc values of polyoxyethylene-containing non-ionic detergents are temperature dependent in that the cmc value becomes higher at lower temperatures (Alexandridis, P. et al, 1994, Nilsson, M. et al, 2007). The cmc of Poloxamer 188 is at least 20-30 mg/ml at 37° C. (Kabanov, A. V. et al, 1995, Alexandridis, P. et al, 1994, Moghimi, S. M. et al, 2004) and increasing to 100 mg/ml at 20° C. (Nakashima, K. et al, 1994). Thus, according to these reports, the cmc of Poloxamer 188 is within the interval of 20-100 mg/ml at 25° C.

Different studies reported that metal ions are involved in the association of the light (80 kDa) and heavy chains (90 kDa) of FVIII (Wang, W. et al, 2003). Therefore, calcium ions (Ca²⁺) are normally present in formulations of FVIII to maintain the association of the complex of both chains.

A variety of stabilizing buffer compositions for FVIII has been described in the art as summarized for example in WO 2010/026186 A1. However, there is little information about suitable buffers stabilizing FVIII at the high protein concentrations needed for subcutaneous administration.

### SUMMARY OF THE INVENTION

The inventors have surprisingly identified that fora buffer suitable for stabilizing FVIII, in particular in a lyophilized state not only the concentration of the bulking agents and cryoprotectants is decisive but also the proportion of these two types of components. In particular, the inventors could show that a weight ratio of cryoprotectants to bulking agents of more than 0.65:1 leads to significantly improved results in long-term storage of FVIII alone or in combination with VWF:

Thus, in a first aspect, the invention relates to a pharmaceutical composition comprising an isolated Factor VIII protein and/or an isolated VWF protein in a stabilizing buffer composition, wherein said pharmaceutical composition is free of albumin and said stabilizing buffer composition comprises cryoprotectants and bulking agents in a weight ratio of more than 0.65:1.

According to a second aspect, the invention relates to a ready-to-use solution for use in the treatment of hemophilia, which is reconstituted from a lyophilized pharmaceutical composition according to the first aspect.

According to a third aspect, the invention relates to the use of a stabilizing buffer as defined in the first aspect for formulating an FVIII protein and/or a VWF protein. Moreover, the inventors have surprisingly found that in addition to the buffer effect the VWF protein has an effect on the in vitro stability of FVIII (see Example 4). Thus, according to a fourth aspect the invention relates to the use of a VWF protein for stabilizing a FVIII protein in vitro.

### FIGURES

FIG. 1 shows the results of a long-term storage experiment of lyophilized compositions of highly concentrated OCTA8 in different stabilizing buffers (N0 to N7) at different temperatures. The storage vials were filled with 2000 IU of OCTA8, then lyophilized and stored at 40° C. (FIG. 1A), 30° C. (FIG. 1B) and 5° C. (FIG. 10), respectively. After the specified storage time, the lyophilisates were reconstituted in 2.5 ml of distilled water and immediately measured for FVIII activity. The columns represent the FVIII activity measured by chromogenic assay. Data sets of OCTA8 in stabilizing buffers N0 to N7 are displayed with the storage time indicated in the legend. The first column of each buffer (storage time ‘month 0’) represents test samples reconstituted and measured right after lyophilisation.

FIG. 2 shows the results of a long-term storage experiment of lyophilized compositions of low concentrated OCTA8 in different stabilizing buffers (N0 to N7) at different temperatures. In FIG. 2A-C the storage temperature is 40° C., 30° C. and 5° C., respectively. The storage vials were filled with 250 IU of OCTA8 and lyophilized. After the specified storage time, the lyophilisate was reconstituted in 2.5 ml of distilled water and directly measured. The columns represent the measured FVIII activity. On the X-axis, the buffer name is identified. The storage time for the individual column is identified in the legend. The first column for each buffer (storage time ‘month 0’) represents the test samples reconstituted and measured right after lyophilisation.

FIG. 3 shows the results of an analysis of the aggregate content in OCTA8 samples stored in the buffers N0-N7 for 3 or 6 months. The percentage of aggregates is represented by the columns shown for each buffer set up.

The percentage of aggregates was determined by size exclusion chromatography.

FIG. 4 shows the results of an analysis of the aggregate content in OCTA8 samples stored in the buffers N0-N7 for 3 or 6 months. The percentage of fragments is represented by the columns shown for each buffer set up. The percentage of fragments was determined by size exclusion chromatography.

FIG. 5 shows the results of an analysis of the monomer content in OCTA8 samples stored in the buffers N0-N7 for 3 or 6 months. The percentage of fragments is represented by the columns shown for each buffer set up. The percentage of monomers was determined by size exclusion chromatography.

FIG. 6 shows the results of a storage experiment of liquid compositions of FVIII proteins alone or in combination with VWF proteins at different temperatures. In FIGS. 6A -C the storage temperature is 5° C., 30° C. and 40° C., respectively. The experimental setup included three samples of OCTA8 alone (OCTA8 CTRL_1, OCTA8 CTRL_2 and OCTA8 CTRL_3), one sample with a wild type FVIII/vWF combination (OCTA8NL), and four samples with OCTA8 in combination with the VWF proteins Seq21 (OCTA8/Seq21), Fragment III (OCTA8/III), OCTA13 (OCTA8/13), OCTA12 (OCTA8/12).

FIG. 7 shows the results of a long-term storage experiment of lyophilized compositions of highly concentrated OCTA8 and OCTA12 in different stabilizing buffers (N4, N11 and N22) at different temperatures after 12 months of storage. In FIG. 7A)-C) the storage temperature is 40° C., 30° C. and 5° C., respectively. The storage vials were filled with a target concentration of 6000 IU OCTA8. OCTA8 was combined with 1.5-1.7 mg of OCTA12 per vial.

### DETAILED DESCRIPTION OF THE INVENTION

Definitions

As used herein, the term “albumin-free” indicates that the composition used contains no albumin in any form, including without limitation Bovine Serum Albumin (BSA) or any form of recombinant albumin.

The transitional term “comprising”, which is synonymous with “including”, “containing”, or “characterized by” is inclusive or open-ended and does not exclude additional, unrecited elements or method steps. The transitional phrase “consisting of” excludes any element, step, or ingredient not specified in the claim, except for impurities ordinarily associated therewith The transitional phrase “consisting essentially of” limits the scope of a claim to the specified materials or steps “and those that do not materially affect the basic and novel characteristic(s)” of the claimed invention. While the term “comprising” does exclude unrecited elements or method steps, such unrecited elements or method steps are not necessarily present. Therefore, any product, composition or method comprising specific recited elements or method steps may be transferred to product, composition or method consisting of the recited elements or method steps.

The term “fragment” as used herein refers to a polypeptide that has an amino-terminal and/or carboxyterminal deletion of one or more amino acids as compared to the native or wild-type protein but where the remaining amino acid sequence is identical to the corresponding positions in the amino acid sequence deduced from a full-length cDNA. Fragments are typically at least 50 amino acids in length. In size-exclusion chromatography experiments, the fraction of “fragments” includes uncomplexed light and heavy chains of FVIII with or without such deletions.

An “ionic strength provider” according to the invention an ionic compound that is present in a composition to increase the ionic strength of the composition.

The term “isolated,” when applied to a nucleic acid or protein, denotes that the nucleic acid or protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. In particular, an isolated gene is separated from open reading frames that flank the gene and encode a protein other than the gene of interest. The term “pure” denotes that a nucleic acid gives rise to one band or a protein that gives rise to one band per chain of the protein in an electrophoretic gel. Particularly, it means that the nucleic acid or protein is at least 85% pure, more preferably at least 95% pure, and most preferably at least 99% pure. The term “histidine-free” according to the invention means that no histidine has been added during the pharmaceutical processing. Does not necessarily mean that the composition is completely “devoid of histidine”. Minor amounts of histidine may follow the bulk drug substance through the manufacturing process.

The term “pharmaceutical composition”, as used herein, refers to a composition comprising pharmaceutically active ingredients and pharmaceutically acceptable excipients providing a therapeutic effect for the treatment and prevention of a disease in a patient.

A “peptide” as used herein may be composed of any number of amino acids of any type, preferably naturally occurring amino acids, which, preferably, are linked by peptide bonds. In particular, a peptide comprises at least 3 amino acids, preferably at least 5, at least 7, at least 9, at least 12, or at least 15 amino acids. Furthermore, there is no upper limit for the length of a peptide. However, preferably, a peptide according to the invention does not exceed a length of 500 amino acids, more preferably it does not exceed a length of 300 amino acids; even more preferably it is not longer than 250 amino acids.

Thus, the term “peptide” includes “oligopeptides”, which usually refer to peptides with a length of 2 to 10 amino acids, and “polypeptides” which usually refer to peptides with a length of more than 10 amino acids.

A “protein” as used herein may contain one or more polypeptide chains. Proteins with more than one polypeptide chain are often expressed as one polypeptide chain from one gene and cleaved post translationally. Thus, the terms “polypeptide” and “protein” are used interchangeably. The polypeptides and proteins as used herein include chemically synthesized proteins as well as naturally synthesized proteins which are encoded by genes. The polypeptides or proteins may be obtained from a natural source, such as human blood or produced in cell culture or transgenic animals as recombinant proteins.

The term “recombinant” when used in reference to a cell, nucleic acid, protein or vector, indicates that the cell, nucleic acid, protein or vector has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell, or express native genes at different levels or under different conditions than found in nature.

The relatedness between two amino acid sequences or between two nucleotide sequences is described by the parameter “sequence identity”. For purposes of the present invention, the degree of sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software

Suite, Rice et a/., 2000, Trends Genet. 16: 276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the no brief option) is used as the percent identity and is calculated as follows:

(Identical Residues×100)/(Length of Alignment−Total Number of Gaps in Alignment).

For purposes of the present invention, the degree of sequence identity between two nucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et a/., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Desoxyribonucleotides×100)/(Length of Alignment−Total Number of Gaps in Alignment)

The terms “similarity” and “similar” as used herein with respect to the definition of a peptide or polynucleotide relate to a specified degree of sequence identity of the amino acid sequence or nucleotide sequence with a reference. A similar amino sequence is taken to include an amino acid sequence that is at least 80%, 85%, 86%, 87%, 88%, 89%, 90%, 91%, 92%, 93%, 94%, 95%, 96%, 97%, 98% or even 99% identical to the subject sequence. Typically, similar sequences will include the same residues in positions that are relevant for the function of the peptide or polynucleotide, such as active site residues or glycosylated amino acids, however though may include any number of conservative amino acid substitutions. A similar nucleotide sequence is taken to include a nucleotide sequence that is at least 80%, 85%, 86%, 87%, 88%, 89%, 90%, 91%, 92%, 93%, 94%, 95%, 96%, 97%, 98% or even 99% identical to the subject sequence.

The abbreviation “% (w/w)”, as used herein, means “weight percentage”, and refers to the weight amount of an excipient of the pharmaceutical composition in relation to the total (overall) weight amount of the excipients in the pharmaceutical composition if nothing else is explicitly stated or obvious under the circumstances.

The Stabilizing Buffer

In a first aspect, the invention relates to a pharmaceutical composition comprising an isolated Factor VIII protein and/or an isolated VWF protein in a stabilizing buffer composition, wherein said pharmaceutical composition is free of albumin and said stabilizing buffer composition comprises cryoprotectants and bulking agents in a weight ratio of more than 0.65:1.

The inventors surprisingly found that the weight ratio of the cryoprotectants and bulking agents in the stabilizing buffer composition has a significant impact on the storage stability of FVIII as determined by FVIII activity. The weight ratio of cryoprotectants and bulking agents of more than 0:65:1 stabilizes FVIII when stored alone or in combination with VWF, although high weight ratios reduce the collapse temperature during lyophilization. Freeze-drying is carried out below the collapse temperature of a composition. Thus, a high collapse temperature is usually favorable for lyophilization as it allows freeze-drying at higher temperatures.

The pharmaceutical composition is albumin-free and, preferably, does not contain other proteins as e.g. trace amounts of plasma proteins. Accordingly, the active components, FVIII and/or VWF, are preferably not derived from plasma but produced recombinantly.

The weight ratio of cryoprotectants and bulking agents may be for example 0.65, 0.67, 0.69, 0.7:1, 0.71:1, 0.73:1; 0.75:1, 0.77:1, 0.79:1, 0.8:1, 0.81:1, 0.83:1, 0.85:1, 0.87:1, 0.89:1, 0.9:1, 0.91:1, 0.93:1, 0.95:1, 0.97:1, 0.99:1, 1:1, 1.01:1, 1.03:1, 1.05:1, 1.07:1, 1.09:1, 1.1:1. According to one embodiment, the weight ratio of the cryoprotectants and bulking agents in the stabilizing buffer composition is in the range of 0.75:1 to 2.2:1. As shown in the Examples, the N7 buffer with a ratio of 2.2:1 under most of the tested conditions stabilizes FVIII and/or VWF. However, under some conditions the stabilizing effect is reduced. Therefore, it is suggested that 2.2:1 represents an upper limit for the ratio of cryoprotectants and bulking agents. The stabilizing effect of the N3 buffer with ratio of cryoprotectants to bulking agents of 0.72:1 is significantly higher than the stabilizing effect of N0 or other buffers with a ratio of 0:6 or less. However, the stabilizing effect of N3 is less pronounced compared to the effect of e.g. N4. Therefore, in a preferred embodiment weight ratio of cryoprotectants to bulking agents is above 0.7:1. The weight ratio may for example be in the range of 0.7:1 to 1.6:1 or in the range of 0.7:1 to 1.4:1. According to one embodiment, the weight ratio of the cryoprotectants and bulking agents in the stabilizing buffer composition is in the range of 0.75:1 to 1.35:1. The buffers with the best stabilizing performances (N4-N6) have weight ratio in the range from 0.90:1 to 1.10:1. Thus, according to one embodiment, the weight ratio of the cryoprotectants and bulking agents in the stabilizing buffer composition is in the range of 0.90:1 to 1.10:1.

The stabilizing buffer of the present invention contains one or more cryoprotectants. A cryoprotectant is a compound present in the formulation to decrease or even prevent loss of protein activity during the freezing and during subsequent storage of the lyophilized product. In case the freezing is a lyophilization process, i.e. includes a drying the stabilizing components are often referred to as cryoprotectants. In the context of this application, both cryo- and lyoprotectant are referred to as cryoprotectants.

The stabilizing buffer may contain, for example, one, two, three or four different cryoprotectants. In case of more than one cryoprotectant, the total mass of cryoprotectants is considered in the determination of the ratio of cryoprotectants and bulking agents. According to one embodiment, the stabilizing buffer contains at least two different cryoprotectants.

The cryoprotectants may be, for example, selected from the group consisting of sucrose, trehalose, lactose, melezitose, arginine and arginine glutamate (glutargin). The listed di- and trisaccharides are able to protect during freezing and as well during drying by replacing hydrogen bonding of sugar to dried protein instead of water. The disaccharides must not be present as pure crystalline structure for this function.

The preferred cryoprotectant is sucrose. As shown in the examples, all buffers with a strong stabilization effect contain sucrose. The stabilizing buffer composition may comprise sucrose in a concentration of more than 23% (w/w). The concentration of sucrose may be, for example, 23% (w/w), 24% (w/w), 25% (w/w), 26% (w/w), 27% (w/w), 28% (w/w), 29% (w/w), 30% (w/w), 31% (w/w), 32% (w/w), 33% (w/w), 34% (w/w), 35% (w/w), 36% (w/w), 37% (w/w), 38% (w/w), 39% (w/w), 40% (w/w), 41% (w/w), 42% (w/w), 43% (w/w), 44% (w/w), 45% (w/w), 46% (w/w), 47% (w/w), 48% (w/w), 49% (w/w), 51% (w/w), 52% (w/w), 53% (w/w), 54% (w/w),55% (w/w), 56% (w/w), 57% (w/w), 58% (w/w), or 59% (w/w). As shown in the examples, stabilizing buffers with a sucrose content of less than 23% (w/w) exhibit a significantly lower stabilization of FVIII and VWF. According to one embodiment, the concentration of sucrose in the stabilizing buffer is in the range of 23 to 59% (w/w). Concentrations above 59% (w/w) may lead to impaired appearance or collapse events of the lyophilization cake. According to one embodiment, the concentration of sucrose in the stabilizing buffer is in the range of 30 to 52% (w/w). According to one embodiment, the concentration of sucrose in the stabilizing buffer is in the range of 37 to 48% (w/w). According to one embodiment, the concentration of sucrose in the stabilizing buffer is in the range of 38 to 41% (w/w). Histidine is a further cryoprotectant often used in the context of FVIII. According to the present invention, the stabilizing buffer is histidine-free.

The stabilizing buffer of the present invention contains one or more bulking agents. The stabilizing buffer may contain, for example, one, two, three or four different bulking agents. A bulking agent according to the invention is referred to as an excipient present in the formulation to provide mechanical support to the lyophilized cake and to increase the dry weight. The bulking agent can either be in a crystalline state, as or in an amorphous state. Suitable bulking agents are sodium chloride, mannitol and glycine. These bulking agents add a crystalline structure or network to the lyophilization cake.

Sodium chloride is the preferred bulking agent. As shown in the examples, all buffers with a strong stabilization effect contain sodium chloride. Moreover, both glycine and NaCl have the additional function of being an ionic strength provider to stabilize the FVIII molecule. This double function minimizes the number of components necessary for an adequate clinical product. Despite the double function, the sodium chloride is counted as a bulking agent.

The stabilizing buffer comprises sodium chloride in a concentration of 16 to 50% (w/w). The concentration may be for example 16% (w/w), 17% (w/w), 18% (w/w), 19% (w/w), 20% (w/w), 21% (w/w), 22% (w/w), 23% (w/w), 24% (w/w), 25% (w/w), 26% (w/w), 27% (w/w), 28% (w/w), 29% (w/w), 30% (w/w), 31% (w/w), 32% (w/w), 33% (w/w), 34% (w/w), 35% (w/w), 36% (w/w), 37% (w/w), 38% (w/w), 39% (w/w), 40% (w/w), 41% (w/w), 42% (w/w), 43% (w/w), 44% (w/w), 45% (w/w), 46% (w/w), 47% (w/w), 48% (w/w), 49% (w/w), or 50% (w/w). Below a concentration of 16% (w/w) the sodium chloride does not provide sufficient mechanical support to the protein ingredients. Above a concentration of 50% (w/w), stability of FVIII at higher storage temperatures is reduced, especially as low concentrations. According to one embodiment, the concentration of sodium chloride is in the range of 28 to 49% (w/w). According to one embodiment, the concentration of sodium chloride in the stabilizing buffer is the range of 32 to 48% (w/w). According to one embodiment, the concentration of sodium chloride in the stabilizing buffer is in the range of 37 to 47% (w/w). According to one embodiment, the concentration of sodium chloride in the stabilizing buffer is in the range of 41 to 46% (w/w). According to one embodiment, the concentration of sodium chloride in the stabilizing buffer is in the range of 43 to 45% (w/w). According to one embodiment, the concentration of sodium chloride in the stabilizing buffer is about 44% (w/w).

According to one embodiment, the stabilizing buffer composition comprises sucrose as cryoprotectant and sodium chloride as bulking agent. According to one embodiment, the sucrose concentration in the stabilizing buffer composition is more than 23% (w/w) and the sodium chloride concentration is in the range of 16 to 50% (w/w).

The stabilizing buffer may contain arginine as a further cryoprotectant. According to one embodiment the concentration of arginine in the stabilizing buffer is less than 16% (w/w). For example, arginine may be present in the stabilizing buffer in a concentration of 0.5% (w/w), 1.0% (w/w), 2.0% (w/w), 3.0% (w/w), 4.0% (w/w), 4.0% (w/w), 5.0% (w/w), 4.5% (w/w), 5.0% (w/w), 5.5% (w/w), 6.0% (w/w), 6.5% (w/w), 7.0% (w/w), 7.5% (w/w), 8.0% (w/w), 8.5% (w/w), 9.0% (w/w), 10.0% (w/w), 11.0% (w/w), 12.0% (w/w), 13.0% (w/w), 14.0% (w/w), 15.0% (w/w), or 15.5% (w/w). According to one embodiment the concentration of arginine in the stabilizing buffer is less than 12% (w/w). According to one embodiment the concentration of arginine in the stabilizing buffer is in the range of 4 to 9% (w/w).

The pH buffering agent according to the invention is preferably a compound with a buffering capacity in the pH range between about pH 5 and 9. The buffering capacity is based on the pKa value of the buffering agent within the said pH interval. Suitable pH buffering agents are e.g. sodium citrate, maleic acid, histidine, and Tris (tris(hydroxymethyl)aminomethane). According to one embodiment, the pH buffering agent is sodium citrate present in an amount to maintain a pH ranging from 6.5 to 7.5. A suitable form of the sodium citrate is the dihydrate salt.

According to one embodiment the sodium citrate is present the range of 0.5 to 18% (w/w). Less than 0.5% (w/w) generally does not provide sufficient buffer strength the composition. A concentration of more than 18% (w/w) is destabilizing due to the calcium chelating properties of the citrate. The concentration may be for example 0.5% (w/w), 0.5% (w/w), 0.7% (w/w), 0.8% (w/w), 0.9% (w/w), 1.0% (w/w), 1.1% (w/w), 1.2% (w/w), 1.4% (w/w), 1.6% (w/w), 1.8% (w/w), 2.0% (w/w), 2.2% (w/w), 2.5% (w/w), 3.0% (w/w), 4.0% (w/w), 5.0% (w/w), 6.0% (w/w), 7.0% (w/w), 8.0% (w/w), 9.0% (w/w), 10.0% (w/w), 12.0% (w/w), 14.0% (w/w), 16.0% (w/w), or 18.0% (w/w). According to one embodiment, the sodium citrate is present in the range of 1.0 to 11% (w/w). According to one embodiment, the concentration of sodium citrate is in the range of 2.2 to 6.5% (w/w). According to one embodiment, the concentration of sodium citrate is in the range of 3 to 5% (w/w). According to one embodiment, the concentration of sodium citrate is about 4% (w/w).

Calcium chloride specifically stabilizes the FVIII molecule. According to one embodiment, the calcium chloride is present the range of 0.2 to 4% (w/w). Less than 0.2% (w/w) is generally not sufficient for stabilization of FVIII. A concentration of more than 4% (w/w) increases propensity of high molecular weight species formation of the VWF fragment. The concentration may be for example 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.3% (w/w), 1.4% (w/w), 1.5% (w/w), 1.6% (w/w), 1.8% (w/w), 2.0% (w/w), 2.2% (w/w), 2.4% (w/w), 2.6% (w/w), 2.8% (w/w), 3.0% (w/w), 3.2% (w/w), 3.4% (w/w), 3.6% (w/w), 3.8% (w/w), or 4.0% (w/w). According to one embodiment, the calcium chloride is present in the stabilization buffer in the range of 0.2 to 4% (w/w). According to one embodiment, the calcium chloride is present in the stabilization buffer in the range of 0.9 to 1.3% (w/w).

A surface-active agent shall mean a compound that adsorbs to surfaces and interfaces and thereby counteracts activity loss of FVIII due to adsorption. This type of activity loss may occur during the entire pharmaceutical processing as well as while handling the reconstituted product prior to and during administration to a patient. Some surface-active agents form micellar aggregates in solution. The critical micelle concentration of a surface-active agent is the concentration above which micelles are formed. The surface-active agent is preferably a non-ionic detergent.

According to one embodiment, the concentration of the non-ionic detergent, in particular Poloxamer 188 in the stabilizing buffer is in the range of 0.1 to 9% (w/w). Less than 0.1% (w/w) generally does not lead to any reduction of FVIII loss due to surface adsorption. A concentration of more than 9% (w/w) increases risk of foam formation during liquid handling. According to one embodiment, the concentration of the non-ionic detergent in the stabilizing buffer is in the range of 1.1 to 8% (w/w).

According to one embodiment, the concentration of the non-ionic detergent in the stabilizing buffer is in the range of 2.2 to 6.3% (w/w).

Non-ionic detergent suitable for the present invention are, for example, selected from the group consisting of Poloxamer 188, Polysorbate 20 and Polysorbate 80. According to one embodiment the non-ionic detergent is Poloxamer 188. Poloxamer 188 is also referred to as Pluronic F-68. The Polysorbates 80 and 20 are described to generally inhibit surface adsorptions of proteins. Poloxamer 188 was found to more specifically inhibit protein surface adsorption. Moreover, Poloxamer 188 appears to be less prone to auto-oxidation compared to Polysorbates, Oxidation could potentially negatively affect the product stability during storage (Maggio et a/. 2012).

The FVIII Protein

The FVIII protein according to the invention is identical to or derived from human full-length wildtype FVIII. This means the FVIII protein contains human full-length FVIII or a functionally active fragment thereof. The functionally active fragment retains the function to activate factor X (to factor Xa) and to bind to VWF. The functionally active FVIII molecule can either be cleaved (dual chain) or uncleaved (single chain). FVIII in humans is encoded by the F8 gene, which comprises 187.000 base pairs in six exons. The transcribed mRNA has a length of 9.029 base pairs and is translated to a protein with 2.351 amino acids from which 19 amino acids are removed. The FVIII molecule in humans is glycosylated with 21 N-linked, and at least 7 O-linked glycans (see Kannicht et al., 2013) and consists of a heavy and a light chain held together by a metal bridge.

After translation, the amino acid chain is cleaved by specific proteases in positions leading to the formation of a heavy chain with about 200 kDa and a light chain with about 80 kDa. The domain organization is typically characterized as A1-A2-B-A3-C1-C2. The light chain is a made-up of domains A3-C1-C2. The heavy chain is in principal composed of the domains A1-A2-B. Heavy chains found in plasma have a heterogeneous composition with molecular weights varying from 90 to 200 kDa. The reason for this are the heterogeneity in its glycosylation, the existence of splice variants and existence of proteolytic products such the B-domain depleted heavy chain A1-A2. The amino acid sequence of the full length FVIII is identified by amino acids 20 to 2.351 of P00451 of UniProtKB, sequence version 1 of Jul. 21, 1986 (in the following UniProtKB P00451.1). According to one embodiment the FVIII protein comprises human full length FVIII identified by amino acids 20 to 2.351 of UniProtKB P00451.1.

Alternatively, the FVIII protein may comprise a functionally active derivative that has an amino acid sequence similar to the human full-length FVIII or a fragment thereof. The functionally active derivative retains the function to activate factor X (to factor Xa) and to bind to VWF.

According to one embodiment, the FVIII protein is FVIII, in which at least part of the B-domain is missing. In this regard, the entire B-domain may be missing. A linker optionally replaces the missing part of the B-domain. The linker sequence has in particular the following amino acids sequence SFSQNSRHQAYRYRRG (SEQ ID NO: 7). An example of a FVIII in which the B-domain is replaced by a linker, is Simoctocog alfa, the active ingredient of Nuwiq® and Vihuma®. Simoctocog alfa has the sequence SEQ ID NO: 1. In the context of this application, Simoctocog alfa is referred to as OCTA8. According to one embodiment, the amino acid sequence of the FVIII protein has an identity of at least 90%, preferably at least 95%, more preferably at least 98% , most preferably 100% to SEQ ID NO: 1. The FVIII protein with this sequence identity to SEQ ID NO: 1 maintains the binding activity to VWF and the ability to form a complex with factor IXa in order to catalyze the conversion of factor X to factor Xa.

According to one embodiment, the amino acid sequence of the FVIII protein has an identity of at least 90%, preferably at least 95%, more preferably at least 98%, most preferably 100% to UniProtKB P00451.1. The FVIII protein with this sequence identity to UniProtKB P00451.1 maintains the binding activity of human full-length FVIII to VWF and the ability to be converted to the activated form, and to form a complex with factor IXa in order to catalyze the conversion of factor X to factor Xa.

In the FVIII protein, the FVIII fragment or derivative may be attached to an attachment moiety. The attachment moiety may be selected from a fusion polypeptide or a conjugation moiety, such as hydroxyethyl starch (HES) or PEG. Suitable fusion polypeptides are antibody fragments in particular as for example Fc-fragments.

The VWF Protein

The VWF protein consists of a VWF peptide and optionally one or more VWF fusion peptides. The VWF peptide is similar to a section of the amino acid sequence of the full-length human wildtype VWF protein as defined by SEQ ID NO: 2. The sequence identity may be at least 95%, at least 98%, or 100%. Moreover, the mature VWF protein is capable of binding to FVIII. According to one embodiment, the section starts with amino acid 764 of SEQ ID NO: 2 and ends with an amino acid of SEQ ID NO: 2 in the range of 1200 to 2000. The mature VWF peptide preferably includes the D′ and D3 domains of VWF. Accordingly, the VWF peptide may start with amino acid 764 of SEQ ID NO: 2 and end with an amino acid of SEQ ID NO: 2 in the range of 1150 to 1400. The ending amino acid may be for example amino acid 1150, 1160, 1170, 1180, 1190, 1200, 1210, 1220, 1230, 1240, 1250, 1260, 1268, 1270, 1280, 1290, 1300, 1310, 1320, 1330, 1340, 1350, 1360, 1370, 1380, 1390, 1400 of SEQ ID NO: 2. The ending amino acid of the section is preferably Histidine 1268 of SEQ ID NO: 2. The VWF peptide may alternatively further include the Al, A2 and A3 domains of VWF. Accordingly, the VWF peptide may start with amino acid 764 of SEQ ID NO: 2 and end with an amino acid of SEQ ID NO: 2 in the range of 1800 to 2000. The ending amino acid may be for example in the range of 1850 to 1950. The ending amino acid may be for example 1810, 1820, 1830, 1840, 1850, 1860, 1870, 1873, 1880, 1890, 1900, 1905, 1910, 1920, 1930, 1940, 1950, 1960, 1970, 1980, 1990, or 2000 of SEQ ID NO: 2. The ending amino acid is preferably in the range of 1850 to 1950.

Exemplary VWF proteins are OCTA12 (SEQ ID NO: 3), Seq21 of WO 2013/160005 A1 (SEQ ID NO: 4), Fragment III of WO 2015/185758 A2 (SEQ ID NO: 5), and OCTA13 (SEQ ID NO: 6).

The VWF protein may contain one or more VWF fusion peptides. For example, the VWF protein may contain 1, 2, 3, 4, 5, 6, or 7 VWF fusion peptides. A VWF fusion peptide may comprise a cluster of O-glycosylated amino acids. According to one embodiment the amino acid sequence of the one or more fusion peptides is similar or identical to a section of human VWF. According to one embodiment, the one or more VWF fusion peptides are identical to amino acids 1238 to 1268 of SEQ ID NO: 2.

According to one embodiment, the amino acid sequence of the VWF protein is similar to SEQ ID NO: 3. The protein with SEQ ID NO: 3 produced in HEK 293F cells is referred to as OCTA12. According to one embodiment, the amino acid sequence of the VWF protein has an identity of at least 90%, preferably at least 95%, more preferably at least 98%, most preferably 100% to SEQ ID NO: 3. The VWF protein with this sequence identity to SEQ ID NO: 3 may maintain the binding activity of OCTA12 to FVIII and/or retain the number of O-glycosylation sites of OCTA12.

According to one embodiment, the amino acid sequence of the VWF protein has an identity of at least 90%, preferably at least 95%, more preferably at least 98%, most preferably 100% to SEQ ID NO: 4. The VWF protein with this sequence identity to SEQ ID NO: 4 may maintain the binding activity of Seq21 to FVIII and/or retain the number of O-glycosylation sites of Seq21.

According to one embodiment, the amino acid sequence of the VWF protein has an identity of at least 90%, preferably at least 95%, more preferably at least 98%, most preferably 100% to SEQ ID NO: 5. The VWF protein with this sequence identity to SEQ ID NO: 5 may maintain the binding activity of Fragment III to FVIII and/or retain the number of O-glycosylation sites of Fragment III.

According to one embodiment, the amino acid sequence of the VWF protein has an identity of at least 90%, preferably at least 95%, more preferably at least 98%, most preferably 100% to SEQ ID NO: 6. The VWF protein with this sequence identity to SEQ ID NO: 6 may maintain the binding activity of OCTA13 to FVIII and/or the number of O-glycosylation sites of OCTA13.

The VWF protein and the FVIII protein may be obtained from plasma or produced recombinantly. The skilled person is aware of methods for purifying VWF and FVIII from plasma. The recombinant VWF protein and the recombinant FVIII protein may be expressed as described in WO-A1-2017/198435. Preferably, the FVIII protein and VWF protein are produced in expression in a human host cell line, in particular a kidney cell line. Preferred human kidney cell lines are HEK cell-lines, in particular the HEK 293 cell line.

The Pharmaceutical Composition

The pharmaceutical composition according to the invention may be in a liquid form or a lyophilized form. Accordingly, the pharmaceutical composition is a solution or a lyophilisate obtained from said solution. Methods of lyophilization are known to the skilled person. For example, the method described in Example 1 may be used.

The total concentration of the components of the stabilizing buffer in a liquid pharmaceutical composition according to the invention is in the range of 10 to 200 g/l. For example, the total concentration of the components of the stabilizing buffer in the liquid pharmaceutical composition may be 10 g/l, 20 g/l, 30 g/l, 40 g/l, 50 g/l, 60 g/l, 70 g/l, 80 g/l, 90 g/l, 100 g/l, 110 g/l, 120 g/l, 130 g/l, 140 g/l, 150 g/l, 160 g/l, 170 g/l, 180 g/l, 190 g/l, or 200 g/l. According to one embodiment, the total concentration of the components of the stabilizing buffer in a liquid pharmaceutical composition according to the invention is in the range of a concentration of 20 to 150 g/l. According to one embodiment, the total concentration of the components of the stabilizing buffer in a liquid pharmaceutical composition according to the invention is in the range of a concentration of 25 to 100 g/l. According to one embodiment, the total concentration of the components of the stabilizing buffer in a liquid pharmaceutical composition according to the invention is in the range of a concentration of 30 to 70 g/l. According to one embodiment, the total concentration of the components of the stabilizing buffer in a liquid pharmaceutical composition according to the invention is in the range of a concentration of 40 to 60 g/l.

The stabilizing buffer is suitable to stabilize the FVIII and/or the VWF protein over broad concentration range. According to one embodiment, the pharmaceutical composition comprises both the FVIII protein and the VWF protein. According to an alternative embodiment, the FVIII protein but not the VWF protein is present in the pharmaceutical composition. According to a further alternative embodiment, the VWF protein but not the VWF protein is present in the pharmaceutical composition. In case the FVIII protein is present in the pharmaceutical composition, the concentration of the Factor VIII protein—as defined by activity tests - is in the range of 100 IU/mL to 25,000 IU/ml. The concentration may be for example 100 IU/ml, 125 IU/ml, 150 IU/ml, 200 IU/ml, 250 IU/ml, 300 IU/ml, 400 IU/ml, 500 IU/ml, 600 IU/ml, 700 IU/ml, 800 IU/ml, 900 IU/ml, 1,000 IU/ml, 1,500 IU/ml, 2,000 IU/ml, 2,500 IU/ml, 3,000 IU/ml, 4,000 IU/ml, 5,000 IU/ml, 6,000 IU/ml, 7,000 IU/ml, 8,000 IU/ml, 9,000 IU/ml, 10,000 IU/ml, 12,000 IU/ml, 14,000 IU/ml, 16,000 IU/ml, 18,000 IU/ml, 20,000 IU/ml, 22,000 IU/mL, 24,000 IU/mL or 25,000 IU/mL. According to one embodiment, the concentration of the Factor VIII protein is in the range 125 IU/mL to 15,000 IU/mL. According to one embodiment, the concentration of the Factor VIII protein is in the range 250 IU/mL to 10,000 IU/mL. According to a further embodiment, the concentration is in the range of 500 IU/mL to 5,000 IU/mL.

For this case, the pharmaceutical composition is a reconstituted liquid solution, the concentration range is 200 IU/mL to 50,000 IU/ml, preferably in the range of 250 IU/mL to 30,000 IU/mL, more preferably in the range of 500 IU/mL to 20,000 IU/mL, more preferably in the range of 1,000 IU/mL to 10,000 IU/mL.

In case the VWF protein is present in the pharmaceutical composition, the concentration of the VWF protein is in the range of 0.01 mg/mL to 100 mg/mL. The concentration of the VWF is determined by Antigen-ELISA. For example, the concentration may be 0.01 mg/mL, 0.02 mg/mL, 0.05 mg/mL, 0.1 mg/mL, 0.2 mg/mL, 0.5 mg/mL, 1.0 mg/mL, 2.0 mg/mL, 3.0 mg/mL, 4.0 mg/mL, 5.0 mg/mL, 7.0 mg/ml, 10 mg/mL, 15 mg/mL, 20 mg/mL, 25 mg/mL, 30 mg/mL, 40 mg/mL, 50 mg/mL, 60 mg/mL, 70 mg/mL, 80 mg/mL, 90 mg/mL, or 100 mg/mL. According to one embodiment the concentration of the VWF protein in the pharmaceutical composition is in the range of 0.1 mg/mL to 10 mg/mL.

According to one embodiment, pharmaceutical composition comprises a FVIII protein and a VWF protein. According to one embodiment, the ratio of the VWF protein to the FVIII protein based on the number of monomers is in the range of 1:1 to 10:1. The ratio may be for example 1:1, 2:1, 3:1, 4:1, 5:1, 6:1, 7:1, 8:1, 9:1, or 10:1. According to one embodiment, the ratio of the VWF protein to the FVIII protein is in the range of 2:1 to 8:1. According to one embodiment, the ratio of the VWF protein to the FVIII protein is in the range of 3:1 to 7:1.

The Ready-To-Use Solution

According to a second aspect, the invention relates to a ready-to-use solution, which is reconstituted from a lyophilized pharmaceutical composition according to the first aspect. The ready-to-use solution may in particular be obtained by the addition of an aqueous diluent. To form the ready-to-use solution it is preferably reconstituted in a volume (e.g. 0.5 ml) that is half the volume that was lyophilized (e.g. 1 ml), leading to a two-fold increase of the concentration of the protein components and excipients. The ready-to-use solution might be prepared in a single device that contains the lyophilized composition and the solvent, i.e. a dual-chamber device.

The ready-to-use solution is for use in medical treatment. The treatment is in particular the treatment of hemophilia A. “Hemophilia” refers to a group of bleeding disorders associated with increased blood clot formation time as compared to blood clot formation time in healthy individuals without hemophilia. Hemophilia includes Hemophilia A, which is a disorder that leads to the absence or decreased levels of FVIII and/or production of defective Factor VIII.

The ready-to-use solution is administered in particular parenterally. Parenteral administration includes intravenous injection, subcutaneous injection, intraperitoneal injection, and intramuscular injection. According to one embodiment the administration route is subcutaneous. According to another embodiment the administration route is intravenous.

Use of the Stabilizing Buffer for Stabilizing FVIII and/or VWF

According to a third aspect the invention relates to the use of a stabilizing buffer as defined in the first aspect for formulating an FVIII protein and/or a VWF protein. In particular, the stabilizing buffer is albumin-free and comprises calcium chloride; sodium citrate; a non-ionic detergent selected from the group consisting of Poloxamer 188, Polysorbate 20, and Polysorbate 80; sucrose in a concentration of more than 8 g/l; NaCl in a concentration 5 to 29 g/l; and arginine in a concentration of less than 8 g/l. According, to one embodiment, the use include lyophilization of the protein in a buffer solution.

The Use of VWF for Stabilizing FVIII

Moreover, the inventors have surprisingly found that in addition to the buffer effect the VWF has an effect on the in vitro-stability of FVIII. As shown in Example 4, the stability in solution of OCTA8 alone or the commercial product Octanate® is lower than the stability of OCTA8 when combined with a VWF protein. Thus, according to a fourth aspect the invention relates to the use of a VWF protein for stabilizing a FVIII protein in vitro. The use comprises mixing the VWF protein and the FVIII protein to form a pharmaceutical composition.

The VWF protein consists of a VWF peptide and optionally one or more VWF fusion peptides. The VWF peptide may have an identity of at least 90% to the amino acid sequence of human wildtype VWF as defined by SEQ ID NO: 2. The sequence identity may also be at least 95%, at least 98%, or 100%. Moreover, the VWF protein is capable of binding to FVIII.

Moreover, shorter fragments of VWF have a significantly stronger effect on the in vitro stability of FVIII as compared to for full length VWF. Thus according to one embodiment, the VWF peptide has an identity of at least 90% to a section the amino acid sequence of human wildtype VWF as defined by SEQ ID NO: 2. The sequence identity may also be at least 95%, at least 98%, or 100%. Moreover, VWF protein is capable of binding to FVIII. According to one embodiment, the section starts with amino acid 764 of SEQ ID NO: 2 and ends with an amino acid of SEQ ID NO: 2 in the range of 1200 to 2200. Moreover, the VWF protein is may be defined as described under the first aspect of the invention (pharmaceutical composition).

The use preferably comprises mixing the VWF with the FVIII protein in a stabilizing buffer as described under the first aspect of the invention. Also the FVIII protein is preferably defined according to this first aspect of the invention above.

When using the VWF protein for stabilizing the FVIII protein, the VWF protein may be used in a ratio to the FVIII protein in the range of 1:1 to 10:1. The ratio may be for example 1:1, 2:1, 3:1, 4:1, 5:1, 6:1, 7:1, 8:1, 9:1, or 10:1. According to one embodiment, the ratio of the VWF protein to the FVIII protein is in the range of 2:1 to 8:1. According to one embodiment, the ratio of the VWF protein to the FVIII protein is in the range of 3:1 to 7:1. A preferably in the range of 2:1 to 8:1, more preferably in the range of 3:1 to 7:1. In the use of the VWF protein for stabilizing the FVIII protein, the composition may be a solution or a lyophilisate.

With the VWF protein, in particular OCTA12, the FVIII protein is stabilized such that the FVIII protein sample maintains its activity in frozen state for at least 6 months. Maintaining the function in frozen state means that the activity of the FVIII protein after frozen storage of the protein solution at -70° C. for the defined time is at least 90% of the starting activity According to one embodiment the FVIII protein sample maintains its activity for at least 12 months. According to one embodiment the FVIII protein sample maintains its activity for at least 24 months. The activity of the FVIII protein after storage in liquid form for 24 h at 25° C. is at least 80% of the starting activity of the FVIII before storage.

Moreover, in association with the VWF protein, the FVIII protein is stabilized such that the FVIII protein sample maintains its activity in lyophilized state for a long time. In particular, the activity of the FVIII protein after storage as a lyophilisate for 24 months at 5° C. or 18 months at 5° C. followed by 6 months at 25° C. and thereafter reconstitution is at least 80% % of the starting activity of the FVIII before storage. The activity of the FVIII protein after storage as a lyophilisate for 12 months at 30° C. and reconstitution is at least 80% of the starting activity of the FVIII before storage.

The invention is further characterized by the following examples.

### EXAMPLES

### Example 1

### Preparation of Stabilizing Buffers

Buffers N0 to N7 were prepared according to the following protocol. All Buffer were prepared several days before use and stored at 2-8° C. until use. Buffer components were weighed in corresponding amounts and dissolved in purified water afterwards. The pH was adjusted to 7.0. All buffers were sterile filtered after manufacturing. The compositions of the buffers are shown in tables 1-10. Table 11 shows the ratio of cryoprotectants and bulking agents in the buffers. Buffers N0 to N2 are comparative buffers.

TABLE 1
Composition of state of the art buffer N0

| | | Weight |
|---|---|---|
| | Concentration | Percentage |
| | [g/l] | [% (w/w)] |
| NaCl | 30 | 0.57 |
| Sucrose | 9 | 0.17 |
| Arginine | 9 | 0.17 |
| Poloxamer 188 | 2 | 0.04 |
| Sodium Citrate | 2 | 0.04 |
| CaCl₂ | 0.5 | 0.01 |
| Total | 52.5 | |

TABLE 2
Composition of comparative buffer N1

| | | Weight |
|---|---|---|
| | Concentration | Percentage |
| | [g/l] | [% (w/w)] |
| NaCl | 15 | 0.53 |
| Sucrose | 9 | 0.32 |
| Arginine | 0 | 0.00 |
| Poloxamer 188 | 2 | 0.07 |
| Citrate | 2 | 0.07 |
| CaCl₂ | 0.5 | 0.02 |
| Sum | 28.5 | |

TABLE 3
Composition of comparative buffer N2

| | | Weight |
|---|---|---|
| | Concentration | Percentage |
| | [g/l] | [% (w/w)] |
| NaCl | 30 | 0.69 |
| Sucrose | 9 | 0.21 |
| Arginine | 0 | 0.00 |
| Poloxamer 188 | 2 | 0.05 |
| Sodium Citrate | 2 | 0.05 |
| CaCl₂ | 0.5 | 0.01 |
| Total | 43.5 | |

TABLE 4
Composition of stabilizing buffer N3

| | | Weight |
|---|---|---|
| | Concentration | Percentage |
| | [g/l] | [% (w/w)] |
| NaCl | 25 | 0.53 |
| Sucrose | 9 | 0.19 |
| Arginine | 9 | 0.19 |
| Poloxamer 188 | 2 | 0.04 |
| Sodium Citrate | 2 | 0.04 |
| CaCl₂ | 0.5 | 0.01 |
| Total | 47.5 | |

TABLE 5
Composition of stabilizing buffer N4

| | | Weight |
|---|---|---|
| | Concentration | Percentage |
| | [g/l] | [% (w/w)] |
| NaCl | 20 | 0.44 |
| Sucrose | 18 | 0.40 |
| Arginine | 3 | 0.07 |
| Poloxamer 188 | 2 | 0.04 |
| Sodium Citrate | 2 | 0.04 |
| CaCl₂ | 0.5 | 0.01 |
| Total | 45.5 | |

TABLE 6
Composition of stabilizing buffer N5

| | | Weight |
|---|---|---|
| | Concentration | Percentage |
| | [g/l] | [% (w/w)] |
| NaCl | 30 | 0.47 |
| Sucrose | 21 | 0.33 |
| Arginine | 9 | 0.14 |
| Poloxamer 188 | 2 | 0.03 |
| Sodium Citrate | 2 | 0.03 |
| CaCl₂ | 0.5 | 0.01 |
| Total | 64.5 | |

TABLE 7
Composition of stabilizing buffer N6

| | | Weight |
|---|---|---|
| | Concentration | Percentage |
| | [g/l] | [% (w/w)] |
| NaCl | 25 | 0.47 |
| Sucrose | 24 | 0.45 |
| Arginine | 0 | 0.00 |
| Poloxamer 188 | 2 | 0.04 |
| Sodium Citrate | 2 | 0.04 |
| CaCl₂ | 0.5 | 0.01 |
| Total | 53.5 | |

TABLE 8
Composition of stabilizing buffer N7

| | | Weight |
|---|---|---|
| | Concentration | Percentage |
| | [g/l] | [% (w/w)] |
| NaCl | 15 | 0.29 |
| Sucrose | 24 | 0.46 |
| Arginine | 9 | 0.17 |
| Poloxamer 188 | 2 | 0.04 |
| Sodium Citrate | 2 | 0.04 |
| CaCl₂ | 0.5 | 0.01 |
| Total | 52.5 | |

TABLE 9
Composition of stabilizing buffer N11

| | | Weight |
|---|---|---|
| | Concentration | Percentage |
| | [g/l] | [% (w/w)] |
| NaCl | 20 | 0.49 |
| Sucrose | 9 | 0.22 |
| Arginine | 6 | 0.15 |
| Poloxamer 188 | 2 | 0.05 |
| Sodium Citrate | 2 | 0.05 |
| CaCl₂ | 0.5 | 0.01 |
| Methionine | 1 | 0.02 |
| Total | 40.5 | |

TABLE 10
Composition of stabilizing buffer N22

| | | Weight |
|---|---|---|
| | Concentration | Percentage |
| | [g/l] | [% (w/w)] |
| NaCl | 25 | 0.49 |
| Sucrose | 21 | 0.22 |
| Arginine | 3 | 0.15 |
| Poloxamer 188 | 2 | 0.05 |
| Sodium Citrate | 2 | 0.05 |
| CaCl₂ | 0.5 | 0.01 |
| Methionine | 1 | 0.02 |
| Total | 54.5 | |

TABLE 11
Ratios of cryoprotectants and bulking
agents in the stabilizing buffers

| | | Bulking |
|---|---|---|
| Buffer | Cryoprotectant | Agent |
| N1 | 0.60 | 1 |
| N2 | 0.60 | 1 |
| N3 | 0.72 | 1 |
| N4 | 1.05 | 1 |
| N5 | 1.00 | 1 |
| N6 | 0.96 | 1 |
| N7 | 2.20 | 1 |
| N11 | 0.75 | 1 |
| N22 | 0.96 | 1 |

### Example 2

### Test of Buffers for Long Term Storage of Highly Concentrated Lyophilized FVIII Compositions

Experimental Procedure

Protein solutions containing 1600-2500 IU OCTA8 in the respective stabilizing buffers N0 to N7 were prepared. Storage vials were filled with 1.5 ml of the protein solutions and lyophilized according to the lyophilization protocol. 18 vials were prepared for each buffer tested. Six vials each of N0 to N7 were used of the storage experiments at the temperatures 4° C., 30° C. and 40° C., i.e. for the time points 1 month, 2 months, 3 months, and 6 months as well as the starting material from time point 0.

The sample representing the starting material was reconstituted immediately after lyophilization and the FVIII activity was determined according to FVIII activity assay protocol.

Lyophilization Protocol

| | Shelf | | | |
|---|---|---|---|---|
| | set point | | | Vacuum |
| Program | temperature | Ramp rate | Hold time | set point |
| step | [° C.] | [° C./min] | [min] | [mbar] |
| Supercooling | −5 | 1 | 30 | N/A |
| Freezing | −50 | 0.8 | 120 | N/A |
| Annealing | −25 | 0.4 | 240 | N/A |
| Freezing | −40 | 0.2 | 120 | N/A |
| | −40 | N/A | 15 | 0.065 |
| Primary drying | −30 | 0.3 | 2520 | 0.065 |
| Secondary drying | 25 | 0.2 | 360 | 0.02 |
| Holding | 5 | N/A | N/A | 0.02 |

Reconstitution: Reconstitution was performed with purified water. Samples were incubated for at least 5 minutes and then resuspended before aliquotation.

FVIII activity assay protocol. A chromogenic activity determination was performed with the BCS-XP system provided by Siemens Healthcare. The assay was carried out according to the description “Factor VIII Chromogenic Assay” and “Applikationsvorschrift für Gerinnungsfaktor VIII mit Faktor VIII Chromogen” provided by Siemens Healthcare protocol.

Results:

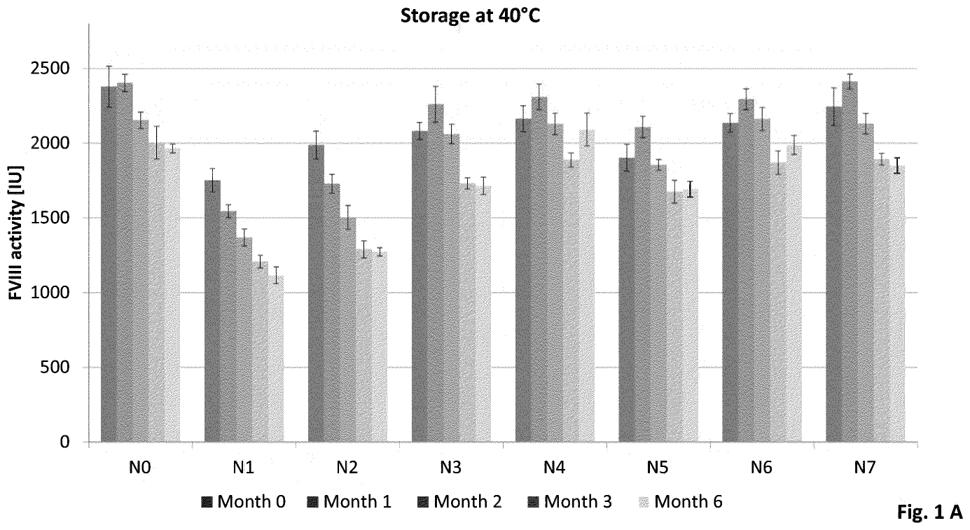

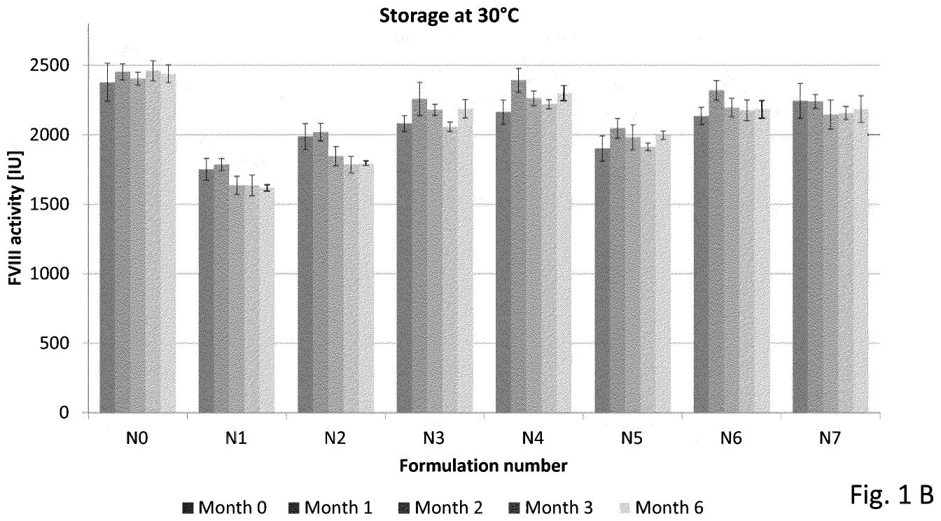

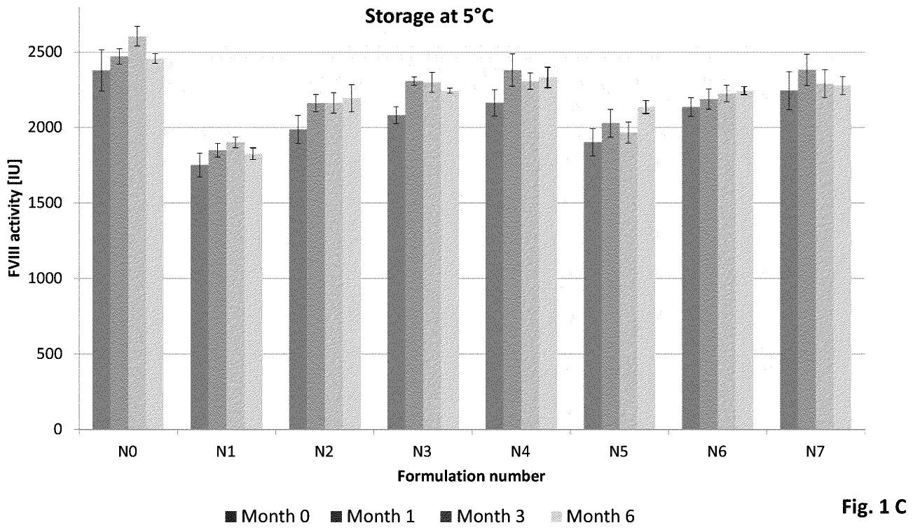

The results are shown in FIG. 1A-C. The increased activity value in the 1 months samples are typical inter-assay variations, e.g. due to variations in the vial filling. Activity results of reconstituted FVIII 2000 IU/vial strengths formulated in buffer compositions N0-N7 demonstrate similar good stability at 5° C. and 30° C. over the monitored time period of six months. At 40° C. storage, FVIII activity losses were detected with best activity values for FVIII formulated in buffer N4.

### Example 3

### Test of Buffers for Long Term Storage of Low Concentrated Lyophilized FVIII Compositions

Protein solutions with 200-313 IU OCTA8 in the respective stabilizing buffers N0 to N7 were prepared. The low concentration experiment is particularly interesting because the lower the protein concentration the less stable is FVIII. Storage vials were filled with 1.5 ml of the protein solution and lyophilized according to the lyophilization protocol. For tested buffer 18 vials were prepared. Six vials of each of N0 to N7 were used of the storage experiments at the temperatures 4° C., 30° C. and 40° C., i.e. for the time points 1 month, 2 months, 3 months, and 6 months as well as the starting material.

The sample representing the starting material reconstituted directly after lyophilization and the FVIII activity was determined according to FVIII activity assay protocol.

Results:

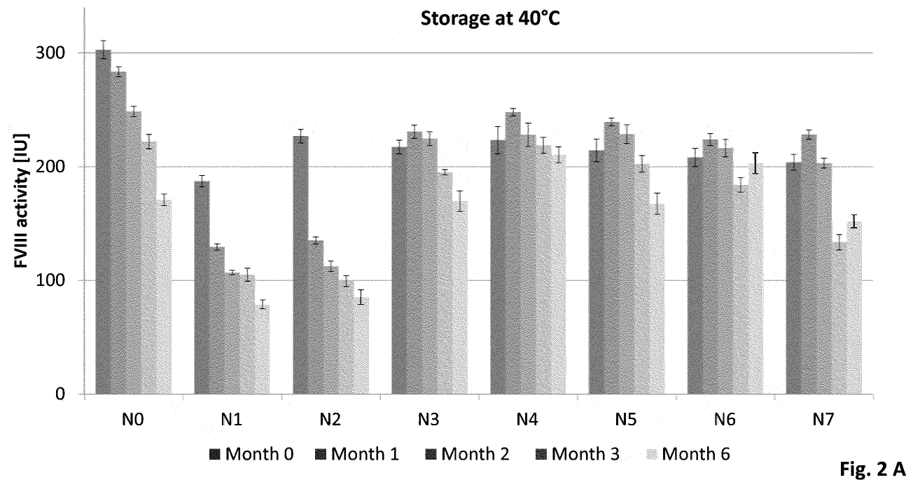

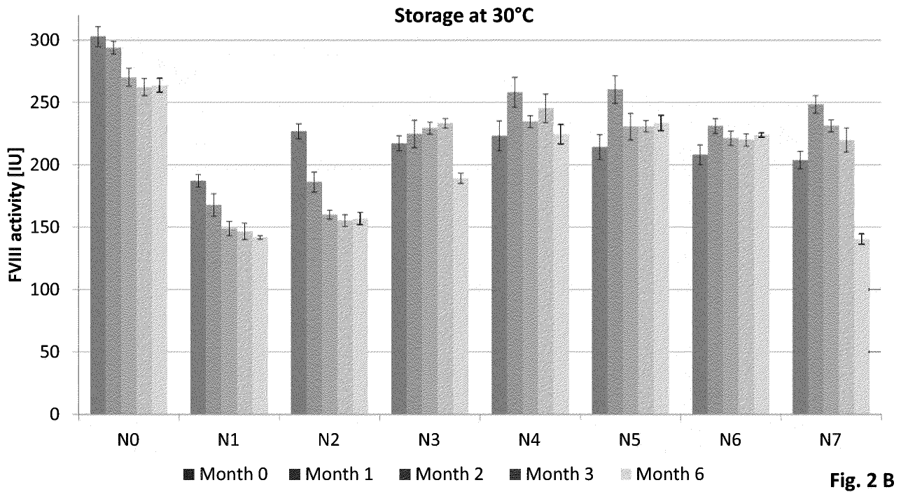

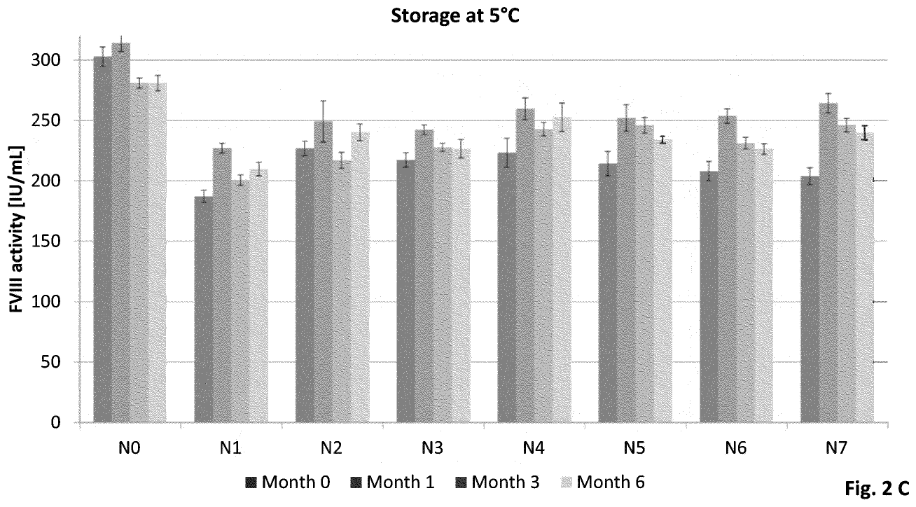

The results are shown in FIG. 2A-C. Activity results of reconstituted FVIII 250 IU/vial strengths formulated in buffer compositions N0-N7 demonstrate similar good stability at 5° C. for all formulations. In contrast, FVIII show an activity loss after 6 months of storage in formulation N0, N1, N2, N3 and N7. At 40° C. the activity losses are more pronounced with best FVIII activity was detected for FVIII formulated in buffer N4.

### Example 4

### Analysis of the Monomer, Fragment and Aggregate Content in the FVIII Compositions After Storage

Experimental Procedure

Monomer, fragment and aggregate analysis was performed by size exclusion chromatography. A 4.6×300 mm LC column of TSKgel SuperSW3000, 4 μm bead size, was connected to a Dionex HPLC system. The mobile phase was composed of 40 mM citrate, 500 mM L-arginine and 2 mM calcium chloride with a pH of 6.5. 20 μL aliquots of the FVIII samples were injected onto the system. The flow rate was set to 0.35 mL/min. UV detection took place at 280 nm.

Results:

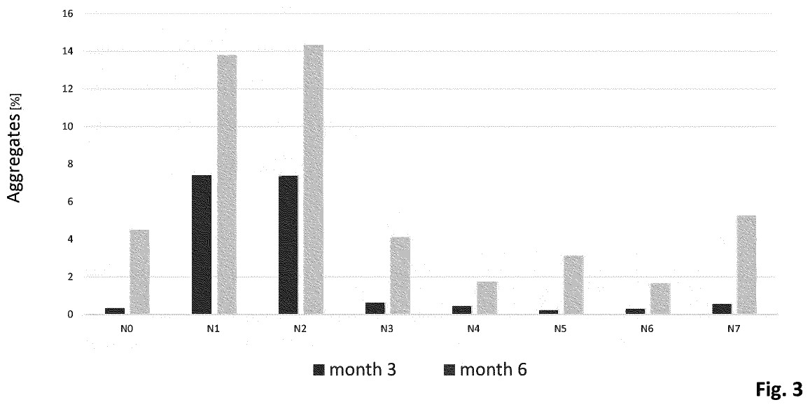

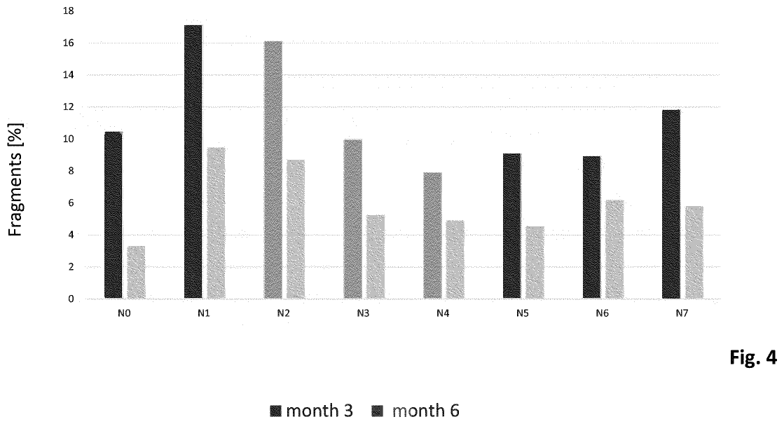

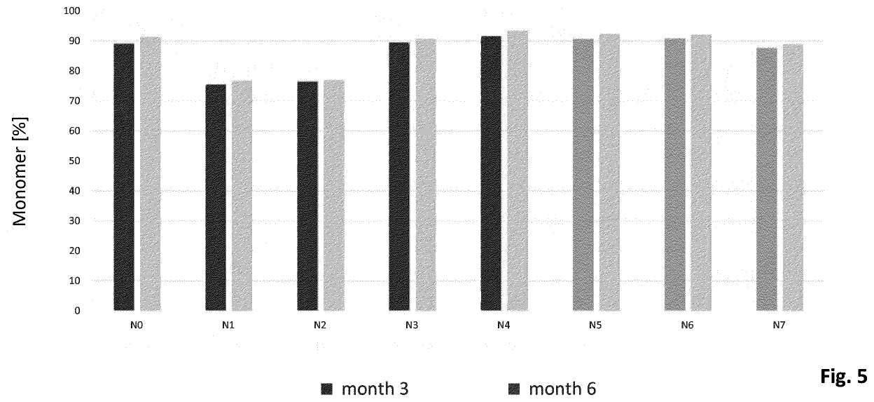

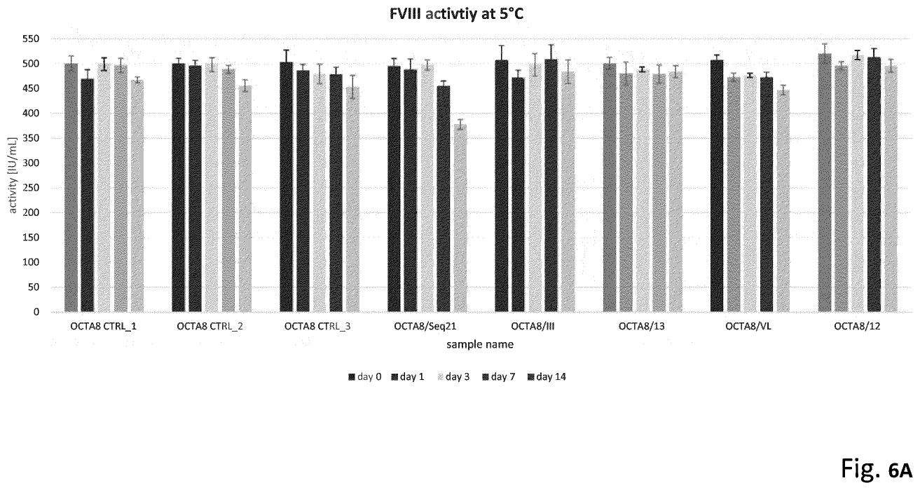

The results are shown in FIGS. 3 to 5. Increased aggregate content was seen in 250 IU/vial samples stored at 30° C. after 6 months of storage. Lowest increases were detected for N4 and N6 and the highest increases were seen in buffers N1 and N2. In contrast, the fragment content decreased from month 3 to month 6. It cannot be ruled out that fragment variants aggregated and, therefore, lead to an increase of the aggregate fraction especially since the monomer content is similar for month 3 and month 6 sample sets.

### Example 4

### Storage Experiment of Liquid Compositions of FVIII Proteins Alone or in Combination with VWF Proteins

Experimental Procedure

This study investigated the stabilizing effect of different vWF fragments or the vWF full-length protein on FVIII. FVIII samples with and without vWF variants were stored in a liquid formulation at 5° C., 30° C. and 40° C. for 14 days at a target FVIII concentration of 500 IU/mL. All vWF fragments or vWF full-length were combined with FVIII (OCTA8) in a 5-fold molar excess in N4 formulation buffer and stored after initial activity determination at the three storage conditions. Samples were taken on days 0, 1, 3, 7 and 14 and immediately analyzed with a chromogenic FVIII activity assay.

Results:

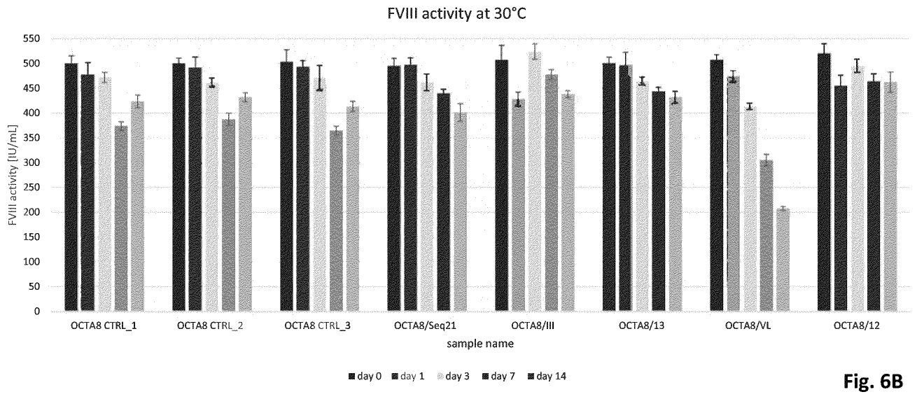

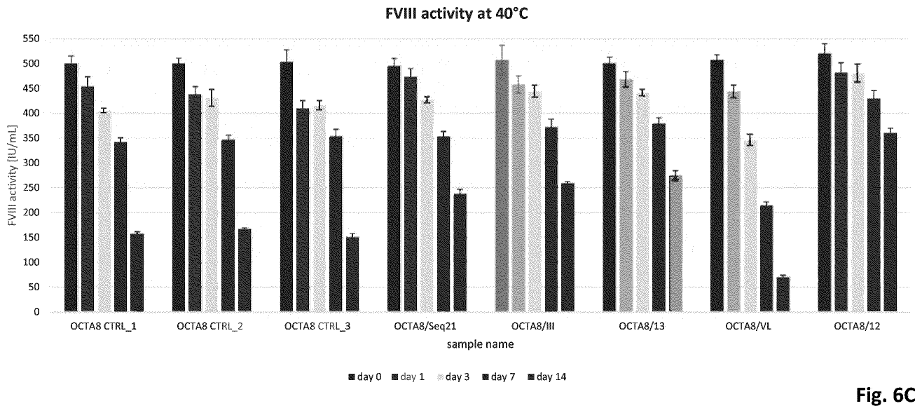

The results are shown in FIG. 6A-C. At 5° C. storage only OCTA8/Seq21 shows a slight drop in activity at day 14. In contrast, significant OCTA8 activity losses can be detected for storage at 30° C. on day 3 for OCTA8/VL and on day 7 for OCTA8/VL and OCTA8 control samples 1-3. A slight decrease in activity is present for all other combinations at this storage condition. At 40° C. storage temperature, the differences in FVIII activity are much more pronounced. Again OCTA8/VL and OCTA8 control samples 1-3 show the highest loss of activity at day 14. The best activity preservation was observed for OCTA101 (OCTA8/12) with 69% compared to the FVIII activity measured on day 0.

### Example 5

### Test of Buffers for Long Term Storage of Highly Concentrated Lyophilized FVIII/vWF Compositions

Experimental Procedure

Three OCTA101 solutions in formulation N4, N11 and N22 with a target concentration of 20000 IU/mL OCTA8 and an OCTA12 concentration of 6.3-6.6 mg/mL were prepared and freeze-dried according to lyophilization protocol of example 1 in 2R vials. Lyophilized OCTA101 Drug Product (DP) with 6000 I U/vial was stored at 5° C.±3° C./ambient relative humidity (rh), 30° C.±2° C./65% rh±5% rh and 40° C.±2° C./75% rh±5% rh. Samples were withdrawn on day 0, month 1, month 2, month 3, month 6, month 9 and month 12 and analyzed by FVIII chromogenic assay.

Results:

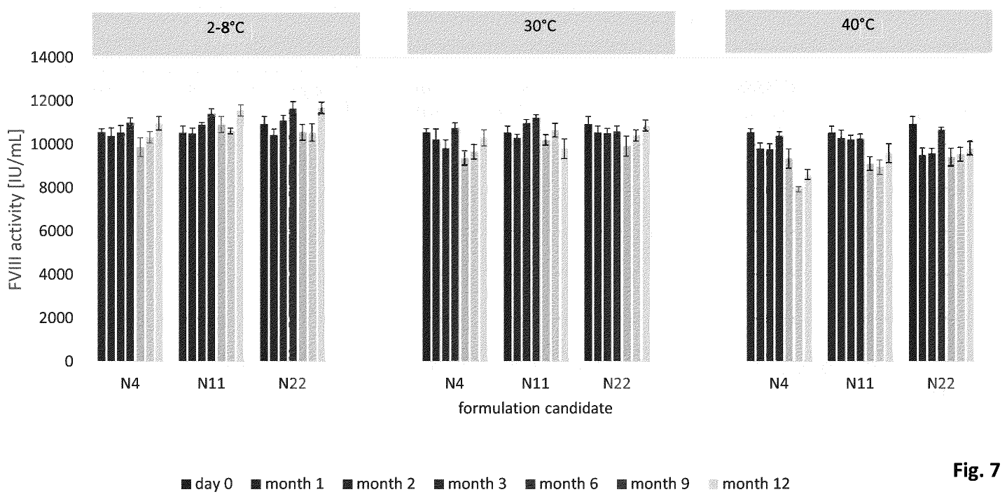

OCTA8 activity results indicate very good stability in all three formulations when present in combination with OCTA12 (FIG. 7). No significant loss of activity could be detected for any of the formulations when stored at 5° C. and 30° C. At 40° C. a less pronounced drop of activity is present for N11 after month 6 and for N4 after month 9. N22 shows best FVIII recovery results in this study.

Many modifications and other embodiments of the invention set forth herein will come to mind to the one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

### REFERENCES

Alexandridis, P. et al, Micellization of Poly(ethylene oxide)-Poly(propylene oxide)-Poly(ethylene oxide) Triblock Copolymers in Aqueous Solutions: Thermodynamics of Copolymer Association, Macromolecules, 27, (1994), 2414-2425

Ewenstein B M, Collins P, Tarantino M D, Negrier C, Blanchette V, Shapiro A D, Baker D, Spotts G, Sensel M, Yi S E, Gomperts E D. Hemophilia therapy innovation development of an advanced category recombinant factor VIII by a plasma/albumin-free method Proceedings of a Special Symposium at the XIXth Congress of the International Society on Thrombosis and Haemostasis; 2004, vol. 41, pg. 1-16.

Kabanov, A. V. et al, Micelle Formation and Solubilization of Fluorescent Probes in Poly(oxyethylene-b-oxypropylene-b-oxyethylene) Solutions, Macromolecules, 28, (1995), 2303-2314

Kannicht, C.; Ramstrom, M.; Kohla, G.; Tiemeyer, M.; Casademunt, E.; Walter, O.; Sandberg, H. (2013): Characterisation of the post-translational modifications of a novel, human cell line-derived recombinant human factor VIII. In Thromb Res. 131 (1), pp. 78-88.

Maggio, E J. Excipients and Food Chem. 3 (2) 2012, 45-53

Manning, M. C, Patel, K. and Borchardt, R. T., Stability of Protein Pharmaceuticals, Pharm. Res., 6 (11), (1989), 903-918

Moghimi, S. M. et al, Biochimica et Biophysica Acta, 2004, 1689, 103-113

Nakashima, K. et al, Fluorescence Studies on the Properties of a Pluronic F68 Micelle, Langmuir, 10, (1994), 658-661

Nilsson, M. et al, Influence of Polydispersity on the Micellization of Triblock Copolymers Investigated by Pulsed Field Gradient Nuclear Magnetic Resonance, Macromolecules, 40, (2007), 8250-8258

Schwegman, J. J., Hardwick, L. M. and Akers, M. J., Practical Formulation and process Development of Freeze-Dried Products, Pharm. Dev. and Techn., 10, (2005), 151-173

Tang, X. and Pikal, M. J., Design of Freeze-Drying Processes for Pharmaceuticals: Practical Advice, Pharm. Res., 21 (2), (2004), 191-200

Vlot A J, Koppelman S J, Meijers J C, Dama C, van den Berg H M, Bouma B N, Sixma J J, Willems G M. Kinetics of factor VIII-von Willebrand factor association. Blood. 1996 1; vol. 87(5); pg. 1809-1816

Wang W., Lyopholisation and development of solid protein pharmaceuticals, Int. J. Pharm., 203, (2000), 1-60

Wang, W., Wang, Y. W. and Kelner, D. N., Coagulation factor VIII: structure and stability (Review), Int. J. Pharm., 259, (2003), 1-15

1. A pharmaceutical composition comprising an isolated Factor VIII protein and/or an isolated VWF protein in a stabilizing buffer composition, wherein said composition is free of albumin and further comprises cryoprotectants and bulking agents in a weight ratio of more than 0.65:1.

2. The pharmaceutical composition according to claim 1, wherein the weight ratio is in the range of 0.65:1 to 2.2:1.

3. The pharmaceutical composition according to claim 1, wherein the cryoprotectants are selected from sucrose, trehalose, glucose, lactose, melezitose, arginine and arginine glutamate, and the bulking agents are selected from glycine, mannitol and sodium chloride.

4. The pharmaceutical composition according to claim 1, wherein:
the stabilizing buffer composition comprises sucrose in a concentration of more than 23% (w/w);
the stabilizing buffer composition comprises sodium chloride in a concentration in the range of 16 to 50% (w/w); and/or
the stabilizing buffer comprises arginine in a concentration of less than 16% (w/w).

5. The pharmaceutical formulation according claim 1, further comprising:
calcium chloride in a concentration in the range of 0.2 to 4% (w/w);
sodium citrate in a concentration in the range of 0.5 to 18% (w/w); and/or
a non-ionic detergent selected from the group consisting of Poloxamer 188, Polysorbate 20 and Polysorbate 80, wherein the concentration of the non-ionic detergent is in the range of 0.1 to 9% (w/w).

6. The pharmaceutical composition according to claim 1, wherein the FVIII protein comprises human full-length FVIII or a functionally active fragment thereof and optionally an attachment selected from a fusion polypeptide and conjugation moiety.

7. The pharmaceutical composition according to claim 6, wherein the functionally active derivative is a fragment of human full-length FVIII, in which at least part of the B-domain is missing.

8. The pharmaceutical composition according to claim 1, wherein the concentration of the Factor VIII protein is in the range of 100 IU/mL to 50,000 IU/mL.

9. The pharmaceutical composition according to claim 1, wherein the VWF protein consists of a VWF peptide and optionally one or more VWF fusion peptides.

10. The pharmaceutical composition according to claim 9, wherein the VWF peptide has an identity of at least 90% to a section of the amino acid sequence of human VWF, wherein the section starts with amino acid 764 of SEQ ID NO: 2 and ends with an amino acid of SEQ ID NO: 2 in the range of 1200 to 2000.

11. The pharmaceutical composition according to claim 10, wherein one or more fusion peptides of the VWF protein comprise a cluster of O-glycosylated amino acids.

12. The pharmaceutical composition according to claim 11, wherein the amino acid sequence of the VWF protein has an identity of at least 90% to SEQ ID NO: 3.

13. The pharmaceutical composition according to claim 1, wherein the pharmaceutical composition is a solution or a lyophilisate.

14. A ready-to-use solution for use in medical treatment, which is reconstituted from a lyophilized pharmaceutical composition according to claim 13 by the addition of an aqueous diluent.

15. The solution of claim 14, wherein the treatment comprises subcutaneous or intravenous administration.

16. A method for stabilizing a FVIII protein in vitro, comprising mixing a VWF protein and a FVIII protein to form a composition, wherein the FVIII protein is defined according to claim 6.

17. The method according to claim 16, wherein the ratio of the VWF protein to the FVIII protein is in the range of 1:1 to 10:1.

18. The method according to claim 16, wherein the composition is a solution or a lyophilisate.

19. The method according to claim 18, wherein the FVIII protein is stabilized such that
a) the activity of the FVIII protein, after frozen storage at −70° C. in a of the protein solution for at least 6 months, is at least 90% of the starting activity of the FVIII before storage;
b) the activity of the FVIII protein, after storage as a lyophilisate for 24 months at 5° C. or 18 months at 5° C. followed by 6 months at 25° C. and thereafter reconstituted, is at least 80% of the starting activity of the FVIII before storage;
c) the activity of the FVIII protein, after storage in liquid form for 24 h at 25° C., is at least 80% of the starting activity of the FVIII before storage; and/or
d) the activity of the FVIII protein, after storage as a lyophilisate for 12 months at 30° C. and thereafter reconstituted, is at least 80% of the starting activity of the FVIII before storage.
